(19) United States
(12) Patent Application Publication
(10) Pub. No.: US 2022/0068073 A1
(43) Pub. Date: Mar. 3, 2022

(54) AUTOMATIC CHANGE MACHINE AND PROGRAM

(71) Applicant: TOSHIBA TEC KABUSHIKI KAISHA, Tokyo (JP)

(72) Inventor: Seiichi Shirato, Mishima Shizuoka (JP)

(21) Appl. No.: 17/238,437

(22) Filed: Apr. 23, 2021

(30) Foreign Application Priority Data
Aug. 25, 2020 (JP) 2020-141975

(51) Int. Cl. G07D 11/16; G07D 11/50; A61L 2/10

(52) U.S. Cl. CPC G07D 11/16; G07D 2211/00; A61L 2/10; G07D 11/50

(57) ABSTRACT

An automatic change machine includes a conveying unit includes a taking-in and conveying unit and a dispensing and conveying unit. The taking-in and conveying unit conveys cash separated one by one to the storage unit. The dispensing and conveying unit conveys the cash separated one by one to the dispensing unit. The sterilizing unit is provided on a conveying path of at least one of the taking-in and conveying unit and the dispensing and conveying unit and sterilizes the cash on a conveying path. The control unit controls conveying speed of the conveying unit to convey the cash at first conveying speed in a depositing and dispensing priority mode for prioritizing depositing and dispensing and convey the cash at second conveying speed lower than the first conveying speed in a sterilization priority mode for prioritizing sterilization of the cash.

### CROSS-REFERENCE TO RELATED APPLICATION

This application is based upon and claims the benefit of priority from Japanese Patent Application No. 2020-141975, filed on Aug. 25, 2020, the entire contents of which are incorporated herein by reference.

### FIELD

Embodiments described herein relate generally to an automatic change machine and a program.

### BACKGROUND

There has been known, for example, a coin depositing and dispensing apparatus and a bill depositing and dispensing apparatus functioning as automatic change machines connected to a POS (Point of Sale System) terminal. A user such as a store clerk who uses these automatic change machines sometimes directly handles cash such as coins and/or bills in payment. Microorganisms, bacteria, viruses, and the like sometimes adhere to cash handled by an unspecified large number of people. Accordingly, for example, a store clerk in a restaurant needs to carry out disinfection work because of a sanitation concern if the store clerk returns to hall work after accounting service or the like. The disinfection work includes work for removing, sterilizing, or inactivating, using soap, alcohol, or the like, at least a part of microorganisms, bacteria, viruses, and the like adhering to fingers that handle cash.

Under such circumstances, there has been disclosed a technique for performing sterilization of microorganisms and bacteria adhering to cash by, for example, irradiating cash stored in a cash box with ultraviolet light.

### DESCRIPTION OF THE DRAWINGS

FIG. 1 is a perspective view illustrating an example of the exterior of an automatic change machine according to an embodiment;

FIG. 2 is a schematic diagram illustrating an example of an internal configuration of the automatic change machine illustrated in FIG. 1;

FIG. 3 is a schematic diagram illustrating an example of the configuration of a sterilizing unit illustrated in FIG. 2;

FIG. 4 is a block diagram illustrating an example of a functional configuration of the automatic change machine illustrated in FIG. 1;

FIG. 5 is a flowchart illustrating an example of processing executed in the automatic change machine illustrated in FIG. 1;

FIG. 6 is a diagram for explaining an example of operation during depositing processing involving sterilizing processing in a depositing and dispensing priority mode of the automatic change machine illustrated in FIG. 1;

FIG. 7 is a diagram for explaining an example of operation during dispensing processing involving the sterilizing processing in the depositing and dispensing priority mode of the automatic change machine illustrated in FIG. 1;

FIG. 8 is a diagram for explaining an example of operation during the sterilizing processing in a sterilization priority mode of the automatic change machine illustrated in FIG. 1;

FIG. 9 is a diagram for explaining another example of the operation during the sterilizing processing in the sterilization priority mode of the automatic change machine illustrated in FIG. 1; and

FIG. 10 is a schematic diagram illustrating another example of the configuration of the sterilizing unit illustrated in FIG. 2.

### DETAILED DESCRIPTION

An aspect of embodiments is to provide, in order to solve the problems described above, an automatic change machine that can appropriately sterilize cash to be stored.

An automatic change machine in an embodiment includes a taking-in unit, a storage unit, a dispensing unit, a conveying unit, a sterilizing unit, and a control unit. The taking-in unit takes cash, which is coins or bills, into an apparatus and separates the taken-in cash one by one. The storage unit stores the cash taken-in from the taking-in unit, separates a number of pieces of the cash of a denomination corresponding to an instruction one by one, and discharges the cash. The dispensing unit dispenses the cash discharged from the storage unit to an outside of the apparatus. The conveying unit includes a taking-in and conveying unit and a dispensing and conveying unit. The taking-in and conveying unit conveys the cash separated one by one by the taking-in unit to the storage unit. The dispensing and conveying unit conveys the cash separated one by one by the storage unit to the dispensing unit. The sterilizing unit is provided on a conveying path of at least one of the taking-in and conveying unit and the dispensing and conveying unit and sterilizes the cash on the conveying path. The control unit controls conveying speed of the conveying unit to convey the cash at first conveying speed in a depositing and dispensing priority mode for prioritizing depositing and dispensing and convey the cash at second conveying speed lower than the first conveying speed in a sterilization priority mode for prioritizing sterilization of the cash.

An automatic change machine and a program according to an embodiment are explained in detail below with reference to the drawings. The invention is not limited to the embodiment explained below.

In the following explanation referring to the drawings, expressions such as up and down, left and right, near side, depth, and front are expressions used assuming that a surface that a user such as a customer or an operator (a store clerk) faces is a front surface.

### First Embodiment

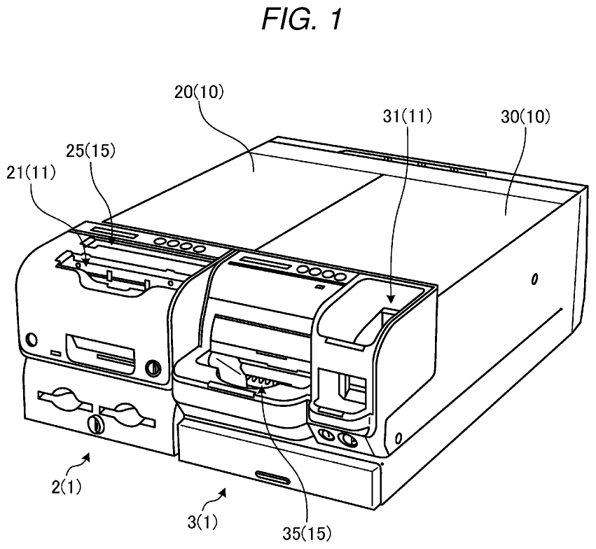

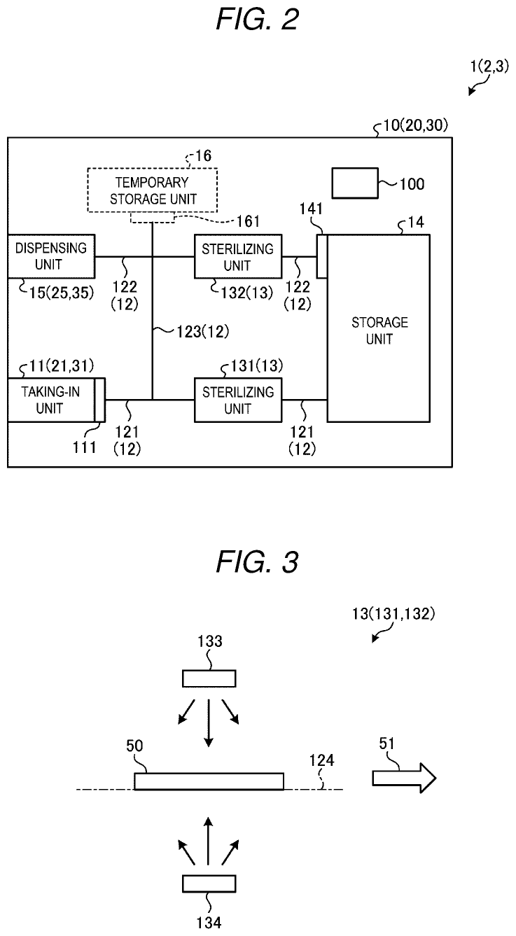

FIG. 1 is a perspective view illustrating an example of the exterior of an automatic change machine 1 according to the embodiment. FIG. 1 illustrates, as an example, a bill depositing and dispensing apparatus 2 functioning as the automatic change machine 1 and a coin depositing and dispensing apparatus 3 functioning as the automatic change machine 1. FIG. 2 is a schematic diagram illustrating an example of an internal configuration of the automatic change machine 1 illustrated in FIG. 1.

In FIG. 1, the bill depositing and dispensing apparatus and the coin depositing and dispensing apparatus 3 respectively including different housings are disposed adjacent to each other. However, not only this, but the automatic change machine 1 according to the embodiment may be the bill depositing and dispensing apparatus 2 or the coin depositing and dispensing apparatus 3 used alone or may be the bill depositing and dispensing apparatus 2 and the coin depositing and dispensing apparatus 3 configured integrally.

In the following explanation, if bills and coins are not distinguished, the bills and the coins are sometimes described as cash or money.

The automatic change machine 1 includes a taking-in unit 11 and a dispensing unit 15 as illustrated in FIG. 1 and FIG. 2.

The taking-in unit 11 is configured to take deposited cash into the inside of a main body 10 while counting the number of pieces of the cash. The taking-in unit 11 is provided on the near side of the automatic change machine 1. The taking-in unit 11 includes a separating mechanism 111. The separating mechanism 111 separates, one by one, the cash deposited from the taking-in unit 11. As the separating mechanism 111, for example, a pair of rollers opposed to each other with a gap of approximately the thickness of a coin or a bill can be used. The pair of rollers can allow an overlapping plurality of pieces of cash to pass through the gap one by one by rotating in the same direction. The separating mechanism 111 only has to be able to separate the overlapping plurality of pieces of cash one by one and may have any configuration. In this way, the taking-in unit 11 takes cash into the main body 10 (an apparatus) and separates the taken-in cash one by one. The taking-in unit 11 supplies the cash separated one by one to a taking-in and conveying unit 121 of a conveying unit 12.

The dispensing unit 15 is configured to dispense cash stored on the inside of the main body 10 to the outside of the main body 10. Specifically, the dispensing unit 15 dispenses, to the outside of the main body 10, cash conveyed from a storage unit 14 by a dispensing and conveying unit 122 of the conveying unit 12 one by one. In other words, the dispensing unit 15 dispenses cash discharged from the storage unit 14 to the outside of the main body 10 (the apparatus). The dispensing unit 15 holds cash to be dispensed to the outside as change until a user takes away the cash. The dispensing unit 15 is provided on the near side of the automatic change machine 1.

A main body 20 of the bill depositing and dispensing apparatus 2 and a main body 30 of the coin depositing and dispensing apparatus 3 correspond to the main body 10 of the automatic change machine 1. A taking-in unit 21 of the bill depositing and dispensing apparatus 2 and a taking-in unit 31 of the coin depositing and dispensing apparatus 3 correspond to the taking-in unit 11 of the automatic change machine 1. A dispensing unit 25 of the bill depositing and dispensing apparatus 2 and a dispensing unit 35 of the coin depositing and dispensing apparatus 3 correspond to the dispensing unit 15 of the automatic change machine 1.

The automatic change machine 1 further includes, as illustrated in FIG. 2, the conveying unit 12 and the storage unit 14. The conveying unit 12 and the storage unit 14 are disposed on the inside of the main body 10.

The conveying unit 12 includes, as illustrated in FIG. 2, the taking-in and conveying unit 121 the dispensing and conveying unit 122, and a circulating and conveying unit 123. The taking-in and conveying unit 121 is configured to convey, to the storage unit 14, cash supplied one by one from the taking-in unit 11. In other words, the taking-in and conveying unit 121 conveys the cash separated one by one by the taking-in unit 11 to the storage unit 14. The dispensing and conveying unit 122 is configured to convey, to the dispensing unit 15, cash supplied one by one from the storage unit 14. In other words, the dispensing and conveying unit 122 conveys cash separated one by one by the storage unit 14 to the dispensing unit 15. The circulating and conveying unit 123 is configured to convey cash on a conveying path of the dispensing and conveying unit 122 onto a conveying path of the taking-in and conveying unit 121. The conveying unit 12 includes, for example, a pair of pulleys and an endless belt stretched at predetermined tension by the pair of pulleys. The endless belt rotates according to driving of one pulley and conveys cash that is in contact with a belt surface. The conveying unit 12 only has to be able to convey cash and may have any configuration.

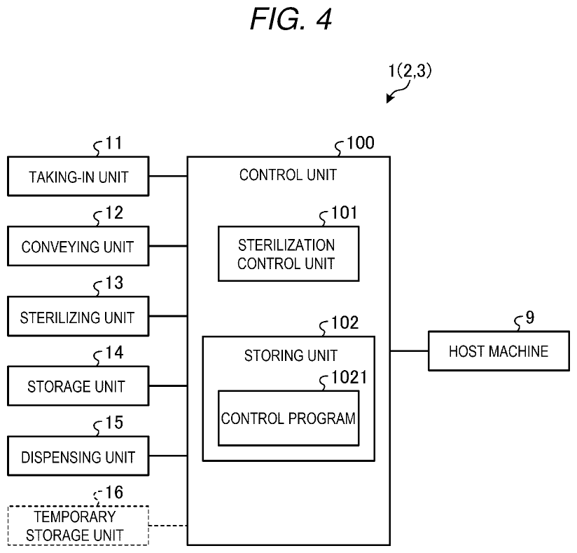

The storage unit 14 includes a plurality of storage spaces for storing cash by denominations. The housing unit 14 counts, for each of the denominations, the number of pieces of cash taken into the inside of the main body 10 by the taking-in unit 11 and stores, for each of the denominations, the cash in the storage space corresponding to the denomination. The number of the plurality of storage spaces is at least a number corresponding to types of the denominations which the automatic change machine 1 handles. The storage unit 14 includes a separating mechanism 141. The separating mechanism 141 separates, one by one, cash discharged from the storage spaces of the storage unit 14. Like the separating mechanism 111 explained above, the separating mechanism 141 only has to be able to separate an overlapping plurality of pieces of cash one by one and may have any configuration. In this way, the storage unit 14 stores the cash taken in from the taking-in unit 11, separates, one by one, the number of pieces of cash of a denomination corresponding to an instruction from a host machine 9 (see FIG. 4), and discharges the cash. The storage unit 14 supplies the cash separated one by one to the dispensing and conveying unit 122 of the conveying unit 12.

The automatic change machine 1 further includes a sterilizing unit 13 as illustrated in FIG. 2. The sterilizing unit 13 is disposed on the inside of the main body 10. The sterilizing unit 13 is configured to sterilize both surfaces of cash conveyed one by one by the conveying unit 12. Specifically, the sterilizing unit 13 includes, as illustrated in FIG. 2, a taking-in and sterilizing unit 131 and a dispensing and sterilizing unit 132. The taking-in and sterilizing unit 131 is provided on the conveying path of the taking-in and conveying unit 121 and configured to sterilize both surfaces of cash conveyed one by one by the taking-in and conveying unit 121. The dispensing and sterilizing unit 132 is provided on the conveying path of the dispensing and conveying unit 122 and configured to sterilize both surfaces of cash conveyed one by one by the dispensing and conveying unit 122. In this way, the sterilizing unit 13 is provided on the conveying paths of the taking-in and conveying unit 121 and the dispensing and conveying unit 122 and sterilizes cash on the conveying paths.

FIG. 3 is a schematic diagram illustrating an example of the configuration of the sterilizing unit 13 illustrated in FIG. 2. The sterilizing unit 13 includes, as illustrated in FIG. 3, an upper-surface irradiating unit 133 and a lower-surface irradiating unit 134. The upper-surface irradiating unit 133 is configured to be capable of irradiating, with ultraviolet light, the upper surface of cash 50 conveyed in the direction of an arrow 51 on a conveying surface 124 of the conveying unit 12. The lower-surface irradiating unit 134 is configured to be capable of irradiating, with the ultraviolet light, the lower surface of the cash 50 conveyed in the direction of the arrow 51 on the conveying surface 124. The conveying surface 124 of the conveying unit 12 is configured to be capable of transmitting the ultraviolet light. The configuration capable of transmitting the ultraviolet light includes using, in a region irradiated by the ultraviolet light, a member transparent to the ultraviolet light, that is, a member having small attenuation of the ultraviolet light. The configuration capable of transmitting the ultraviolet light includes not disposing, in the region irradiated by the ultraviolet light, a member that blocks the ultraviolet light and a member that greatly attenuates the ultraviolet light. As an example, a part or the entire endless belt of the conveying unit 12 is configured by a member having transmittance to the ultraviolet light. As another example, a hole for causing the ultraviolet light to pass is provided in a part such as the center of the endless belt of the conveying unit 12.

Each of the upper-surface irradiating unit 133 and the lower-surface irradiating unit 134 is configured to be capable of radiating light having a wavelength in a predetermined ultraviolet region (hereinafter referred to as ultraviolet light). As each of the upper-surface irradiating unit 133 and the lower-surface irradiating unit 134, for example, an LED (Light Emitting Diode) can be used. Each of the upper-surface irradiating unit 133 and the lower-surface irradiating unit 134 only has to be able to radiate ultraviolet light in a predetermined wavelength region and is not limited to the LED. A mercury lamp or the like may be used.

The ultraviolet light used by the sterilizing unit 13 may include a wavelength of any one of UV-A (wavelength: 320 to 380 nm), UV-B (wavelength: 280 to 320 nm), and UV-C (wavelength: 200 to 280 nm). However, the ultraviolet light used by the sterilizing unit 13 is preferably ultraviolet light in a wavelength region including the wavelength of the UV-C. The ultraviolet light used by the sterilizing unit 13 is more preferably ultraviolet light in a wavelength region including a wavelength of 250 to 260 nm. The ultraviolet light used by the sterilizing unit 13 is still more preferably ultraviolet light in a wavelength region including a wavelength of 253.7 nm.

As radiation intensity of the ultraviolet light by the sterilizing unit 13, intensity requested for sterilization corresponding to, for example, types of microorganisms, bacteria, and viruses, which are targets of sterilizing processing, only has to be set as appropriate according to the distance between each of the upper-surface irradiating unit 133 and the lower-surface irradiating unit 134 and the conveying surface 124.

A region on the conveying surface 124 irradiated with the ultraviolet light by the upper-surface irradiating unit 133 and a region on the conveying surface 124 irradiated with the ultraviolet light by the lower-surface irradiating unit 134 may not coincide.

In each of the taking-in and sterilizing unit 131 and the dispensing and sterilizing unit 132, the upper-surface irradiating unit 133 and the lower-surface irradiating unit 134 may be integrally configured. For example, the upper-surface irradiating unit 133 and the lower-surface irradiating unit 134 configured integrally may have a size and a shape capable of irradiating regions on both sides of the conveying surface 124 on the conveying path with the ultraviolet light at a time. Alternatively, the upper-surface irradiating unit 133 and the lower-surface irradiating unit 134 configured integrally may have a common light source and an optical system that deflects ultraviolet light emitted from the light source and may be configured to be capable of irradiating the regions on both the sides of the conveying surface 124 on the conveying path with the ultraviolet light.

The taking-in and sterilizing unit 131 and the dispensing and sterilizing unit 132 may be integrally configured. For example, each of the upper-surface irradiating unit 133 and the lower-surface irradiating unit 134 may have a size and a shape capable of irradiating both of a region on the conveying path of the taking-in and conveying unit 121 and a region on the conveying path of the dispensing and conveying unit 122 with the ultraviolet light at a time. Alternatively, each of the upper-surface irradiating unit 133 and the lower-surface irradiating unit 134 may have a common light source and an optical system that deflects ultraviolet light emitted from the light source and may be configured to be capable of irradiating both the regions on the conveying path of the taking-in and conveying unit 121 and the conveying path of the dispensing and conveying unit 122 with the ultraviolet light.

One of the taking-in and sterilizing unit 131 and the dispensing and sterilizing unit 132 may not be provided in the sterilizing unit 13. That is, the sterilizing unit 13 is provided on the conveying path of at least one of the taking-in and conveying unit 121 and the dispensing and conveying unit 122 and sterilizes the cash 50 on the conveying path.

In this embodiment, sterilizing means reducing the number of microorganisms, bacteria, and viruses or detoxicating microorganisms, bacteria, and viruses. That is, the sterilizing in this embodiment includes removing and/or annihilating at least a part of microorganisms and bacteria. The sterilizing in this embodiment includes removing and/or inactivating at least a part of viruses.

The automatic change machine 1 further includes a control unit 100 as illustrated in FIG. 2. The control unit 100 is disposed on the inside of the main body 10 of the automatic change machine 1. The control unit 100 includes a processor, a memory, an I/O device control unit, and a communication I/F (all of which are not illustrated). As the processor, for example, a CPU (Central Processing Unit) is used. As the memory, for example, a RAM (Random Access Memory) and a ROM (Read Only Memory) are used. The RAM is a volatile memory that is used as a working memory and in which data is stored if the processor executes arithmetic processing. The ROM is a nonvolatile memory that stores programs such as a control program 1021 (see FIG. 4) to be executed by the processor and data such as parameters.

The processor is not limited to the CPU. Another processor such as a GPU (Graphics Processing Unit), an ASIC (Application Specific Integrated Circuit), or an FPGA (Field Programmable Gate Array) may be used. The memory is not limited to the RAM and the ROM. Another nonvolatile memory such as a HDD (Hard Disk Drive), an SSD (Solid State Drive), or a flash memory may be used.

FIG. 4 is a block diagram illustrating an example of a functional configuration of the automatic change machine 1 illustrated in FIG. 1. As illustrated in FIG. 4, the control unit 100 is communicably connected to the taking-in unit 11, the conveying unit 12, the sterilizing unit 13, the storage unit 14, the dispensing unit 15, and the host machine 9. The host machine 9 is a computer such as a POS (Point of Sale System) terminal mounted on various registers. As an example, the host machine 9 can be placed on an upper part of the main body 10 of the automatic change machine 1.

The control unit 100 controls the units of the automatic change machine 1 according to various programs stored in the ROM and the like. The processor of the control unit 100 executes various programs loaded in the RAM to thereby store a counting result in the memory or transmit the counting result to the host machine 9. The processor of the control unit 100 executes the various programs loaded in the RAM to thereby receive instructions such as a depositing instruction, a dispensing instruction, a sterilizing instruction, and a recounting instruction from the host machine 9.

As an example, the processor of the control unit 100 executes the control program 1021 loaded in the RAM to thereby realize functions of a sterilization control unit 101 and a storing unit 102.

The sterilization control unit 101 controls the sterilizing processing in the automatic change machine 1. The sterilizing processing includes sterilizing processing in a depositing and dispensing priority mode and sterilizing processing in a sterilization priority mode. The depositing and dispensing priority mode is, for example, a mode executed while a store is open. The sterilization priority mode is a mode executed at timing when a time for the sterilizing processing can be sufficiently secured such as before the store is opened or after the store is closed. In the depositing and dispensing priority mode for prioritizing depositing and dispensing, the sterilization control unit 101 controls conveying speed of the conveying unit 12 to convey cash at first conveying speed. In the sterilization priority mode for prioritizing sterilization of cash, the sterilization control unit 101 controls the conveying speed of the conveying unit 12 to convey cash at second conveying speed lower than the first conveying speed.

The storing unit 102 stores the control program. 1021 for controlling the operation of the automatic change machine 1. The storing unit 102 stores various parameters relating to the operation of the automatic change machine 1. For example, the storing unit 102 stores the conveying speed for cash by the conveying unit 12 in the respective modes.

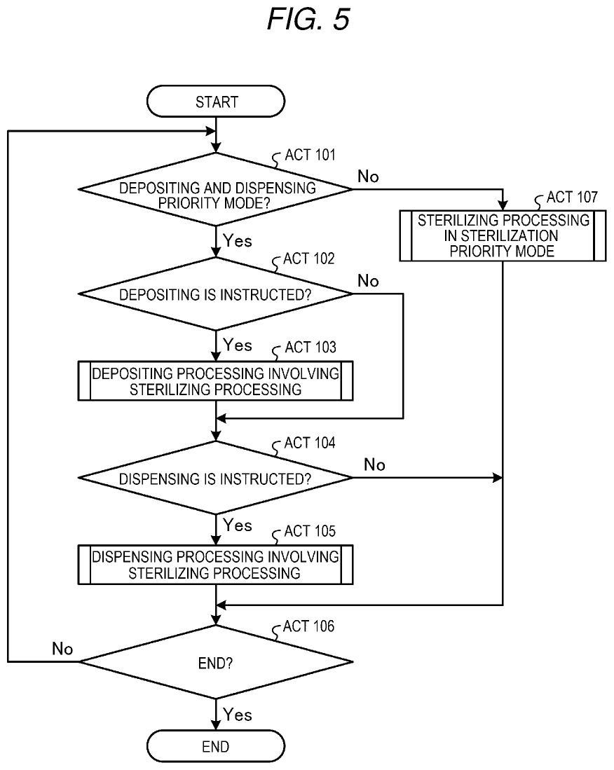

A flow of processing executed in the automatic change machine 1 according to the embodiment is explained with reference to the drawings. FIG. 5 is a flowchart illustrating an example of processing executed in the automatic change machine 1 illustrated in FIG. 1.

The sterilization control unit 101 determines whether the automatic change machine 1 operates in the depositing and dispensing priority mode (ACT 101). For example, if not receiving the sterilizing instruction from the host machine 9, the sterilization control unit 101 determines that the automatic change machine 1 operates in the depositing and dispensing priority mode. For example, if receiving the sterilizing instruction from the host machine 9, the sterilization control unit 101 determines that the automatic change machine 1 does not operate in the depositing and dispensing priority mode, that is, operates in the sterilization priority mode.

The sterilization control unit 101 may determine, for example, based on the reception of the depositing instruction or the dispensing instruction from the host machine 9, that the automatic change machine 1 operates in the depositing and dispensing priority mode.

Depositing and Dispensing Priority Mode

If determining that the automatic change machine 1 operates in the depositing and dispensing priority mode (Yes in ACT 101), the sterilization control unit 101 starts operation in the depositing and dispensing priority mode (ACT 102 to ACT 105).

The sterilization control unit 101 determines, for example, according to whether the depositing instruction is received from the host machine 9, whether to execute depositing processing (ACT 102). If determining to execute the depositing processing (Yes in ACT 102), the sterilization control unit 101 executes the depositing processing involving the sterilizing processing (ACT 103).

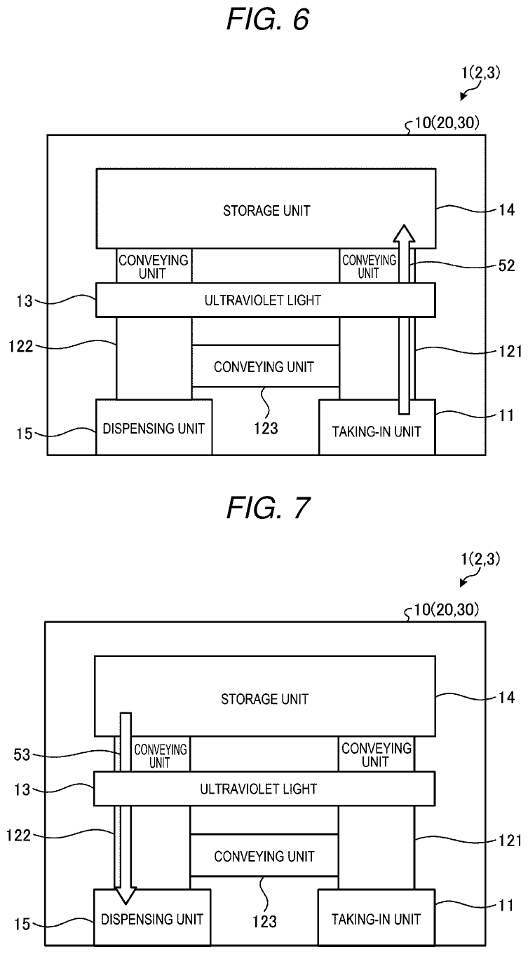

FIG. 6 is a diagram for explaining an example of operation during the depositing processing involving the sterilizing processing in the depositing and dispensing priority mode of the automatic change machine 1 illustrated in FIG. 1. In FIG. 6, an arrow 52 indicates a flow of cash in the depositing processing. In the depositing processing, the sterilization control unit 101 takes cash into the inside of the main body 10 with the taking-in unit 11 and separates the taken-in cash one by one. The sterilization control unit 101 conveys, with the taking-in and conveying unit 121, the cash separated one by one to the storage unit 14. The sterilization control unit 101 irradiates, with the sterilizing unit 13, both sides of the cash conveyed one by one with ultraviolet light.

In the depositing processing, the sterilization control unit 101 sets conveying speed of the taking-in and conveying unit 121 to speed for prioritizing taking-in of cash. The conveying speed of the taking-in and conveying unit 121 corresponds to a sterilization time of cash by the sterilizing unit 13 during the depositing processing.

If not determining to execute the depositing processing (No in ACT 102), the sterilization control unit 101 determines, for example, according to whether the dispensing instruction is received from the host machine 9, whether to execute dispensing processing (ACT 104). If determining to execute the dispending processing (Yes in ACT 104), the sterilization control unit 101 executes the dispensing processing involving the sterilizing processing (ACT 105).

FIG. 7 is a diagram for explaining an example of operation during the dispensing processing involving the sterilizing processing in the depositing and dispensing priority mode of the automatic change machine 1 illustrated in FIG. 1. In FIG. 7, an arrow 53 indicates a flow of cash in the dispensing processing. In the dispensing processing, the sterilization control unit 101 discharges the number of pieces of cash of a denomination corresponding to the dispensing instruction one by one with the storage unit 14. The sterilization control unit 101 conveys, with the dispensing and conveying unit 122, the cash separated one by one to the dispensing unit 15. The sterilization control unit 101 irradiates, with the sterilizing unit 13, both sides of the cash conveyed one by one with ultraviolet light.

In the dispensing processing, the sterilization control unit 101 sets conveying speed of the dispensing and conveying unit 122 to speed for prioritizing dispensing of cash. The conveying speed of the dispensing and conveying unit 122 corresponds to a sterilization time of cash by the sterilizing unit 13 during the dispensing processing.

Sterilization Priority Mode

If determining that the automatic change machine 1 operates in the sterilization priority mode (No in ACT 101), the sterilization control unit 101 starts the sterilizing processing in the sterilization priority mode (ACT 107).

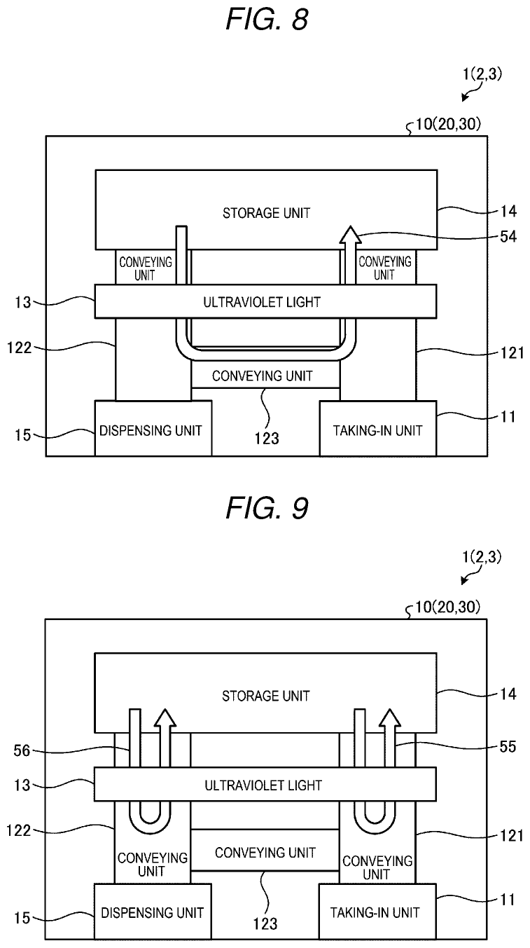

FIG. 8 is a diagram for explaining an example of operation during the sterilizing processing in the sterilization priority mode of the automatic change machine 1 illustrated in FIG. 1. In FIG. 8, an arrow 54 indicates a flow of cash in the sterilizing processing in the sterilization priority mode. In the sterilizing processing in the sterilization priority mode, the sterilization control unit 101 discharges the number of pieces of cash of a denomination corresponding to the sterilizing instruction one by one with the storage unit 14. The sterilization control unit 101 conveys the cash separated one by one to the storage unit 14 with the dispensing and conveying unit 122, the circulating and conveying unit 123, and the taking-in and conveying unit 121. The sterilization control unit 101 irradiates, with the sterilizing unit 13, both sides of the cash conveyed one by one with ultraviolet light.

In the sterilizing processing in the sterilization priority mode, a sterilizing operation by the sterilizing unit 13 only has to be carried out on at least one of a taking-in side and a dispensing side.

In the sterilizing processing in the sterilization priority mode, the sterilization control unit 101 sets the conveying speed of the conveying unit 12 to speed for prioritizing sterilization of cash. The conveying speed of the conveying unit 12 corresponds to a sterilization time of cash by the sterilizing unit 13 during the sterilizing processing in the sterilization priority mode. The speed for prioritizing sterilization of cash, that is, the conveying speed in the sterilization priority mode is lower than the conveying speed in the depositing and dispensing priority mode. Therefore, the sterilization time of cash in the sterilization priority mode is longer than the sterilization time in the depositing and dispensing priority mode. Specifically, the speed for prioritizing sterilization of cash only has to be set as appropriate based on radiation intensity of ultraviolet light by the sterilizing unit 13, the distance between the sterilizing unit 13 and the conveying surface 124, a time requested for sterilization corresponding to, for example, types of microorganisms, bacteria, and viruses, that are targets of sterilizing processing, and the like.

The flow illustrated in FIG. 5 proceeds to processing in ACT 106 if it is not determined to execute the dispensing processing (No in ACT 104) and after the sterilizing processing in the sterilization priority mode (ACT 107) ends. The sterilization control unit 101 determines whether to end the processing (ACT 106). For example, if receiving a power-off instruction for turning off a power supply of the automatic change machine 1 from the host machine 9, the sterilization control unit 101 determines to end the processing. If it is not determined to end the processing (No in ACT 106), the flow illustrated in FIG. 5 returns to the processing in ACT 101 and repeats the processing in ACT 101 to ACT 107. If it is determined to end the processing (Yes in ACT 106), the flow illustrated in FIG. 5 ends.

A user such as a store clerk who uses the automatic change machine 1 sometimes directly handles cash such as coins and/or bills in payment. Microorganisms, bacteria, viruses, and the like sometimes adhere to cash handled by an unspecified large number of people. Accordingly, for example, a store clerk in a restaurant needs to carry out disinfection work because of a sanitation concern if the store clerk returns to hall work after accounting service or the like. The disinfection work includes work for removing, sterilizing, or inactivating, using soap, alcohol, or the like, at least a part of microorganisms, bacteria, viruses, and the like adhering to fingers that handle cash.

Under such circumstances, the automatic change machine 1 in the embodiment includes the taking-in unit 11, the storage unit 14, the dispensing unit 15, the conveying unit 12, the sterilizing unit 13, and the control unit 100. The conveying unit 12 includes the taking-in and conveying unit 121 and the dispensing and conveying unit 122. The taking-in and conveying unit 121 conveys cash separated one by one by the taking-in unit 11 to the storage unit 14. The dispensing and conveying unit 122 conveys the cash separated one by one by the storage unit 14 to the dispensing unit 15. The sterilizing unit 13 is provided on the conveying path of at least one of the taking-in and conveying unit 121 and the dispensing and conveying unit 122 and sterilizes the cash on the conveying path. The sterilization control unit 101 of the control unit 100 controls the conveying speed of the conveying unit 12 to convey the cash at the first conveying speed in the depositing and dispensing priority mode for prioritizing depositing and dispensing and convey the cash at the second conveying speed lower than the first conveying speed in the sterilization priority mode for prioritizing sterilization of the cash.

With this configuration, in the automatic change machine 1, it is possible to sterilize cash during depositing and dispensing of the cash (the sterilizing processing in the depositing and dispensing priority mode). After storing cash, concerning the cash stored in the storage unit 14, it is possible to automatically periodically repeat storage and conveyance and maintain a sterilized state (the sterilizing processing in the sterilization priority mode). Consequently, it is possible to obtain an effect of improving sanitation if the user handles cash. In other words, it is possible to eliminate the sanitation concern if the user handles cash. In this way, with the technique according to the embodiment, it is possible to appropriately sterilize cash to be stored.

The automatic change machine 1 according to the embodiment irradiates, with ultraviolet light, both sides of cash conveyed by the conveying unit 12 and separated one by one. Consequently, unlike when cash on the inside of the storage unit 14 is irradiated with the ultraviolet light, it is possible to sterilize both the surfaces of the cash in a state in which the cash does not overlap. Therefore, it is possible to more appropriately maintain a sterilized state of the cash.

### Second Embodiment

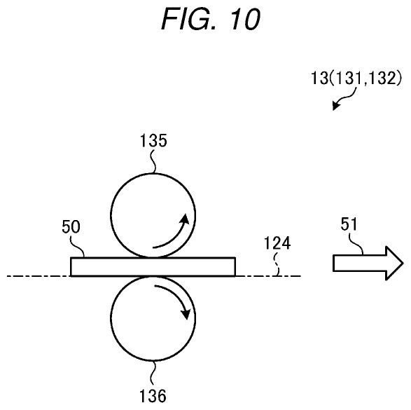

In the first embodiment, concerning the cash stored in the storage unit 14, the sterilizing processing in the sterilization priority mode is performed using the dispending and conveying unit 122, the circulating and conveying unit 123, and the taking-in and conveying unit 121. However, the operation during the sterilizing processing is not limited to this. FIG. 9 is a diagram for explaining another example of the operation during the sterilizing processing in the sterilization priority mode of the automatic change machine 1 illustrated in FIG. 1. In FIG. 9, arrows 55 and 56 indicate a flow of cash in the sterilizing processing in the sterilization priority mode.

In the example illustrated in FIG. 9, the automatic change machine 1 may not include the circulating and conveying unit 123. On the other hand, the storage unit 14 supplies cash separated one by one to the taking-in and conveying unit 121 in addition to the dispensing and conveying unit 122. Each of the taking-in and conveying unit 121 and the dispensing and conveying unit 122 is configured to be capable of reversing a conveying direction.

The sterilization control unit 101 discharges, one by one, the number of pieces of cash of a denomination corresponding to a sterilizing instruction with the storage unit 14. The sterilization control unit 101 conveys, with the taking-in and conveying unit 121 and the dispensing and conveying unit 122, the cash separated one by one further to the side of the taking-in unit 11 and the dispensing unit 15 than the sterilizing unit 13. At this time, a conveying direction of the taking-in and conveying unit 121 is reversed from the conveying direction in the first embodiment. The sterilization control unit 101 irradiates, with the sterilizing unit 13, both sides of the cash conveyed one by one with ultraviolet light. Thereafter, the sterilization control unit 101 conveys the cash sterilized by the sterilizing unit 13 to the storage unit 14 and stores the cash in the storage unit 14. At this time, a conveying direction of the dispensing and conveying unit 122 is reversed from the conveying direction in the first embodiment.

The cash sterilized using the taking-in and conveying unit 121 and the cash sterilize using the dispensing and conveying unit 122 may be the same denomination or may be different denominations.

With this configuration, since the circulating and conveying unit 123 may not be provided, it is possible to reduce the automatic change machine 1 in size and improve flexibility of disposition of internal components. Since a conveyance amount of cash by the conveying unit 12 can be increased, it is possible to reduce a time required for the sterilizing processing.

### Third Embodiment

In the embodiments explained above, concerning the cash stored in the storage unit 14, the sterilizing processing is automatically performed in the sterilization priority mode. However, not only this, but the cash set as a target of the sterilizing processing in the sterilization priority mode may be put in the taking-in unit 11 by a user such as a store clerk. In this case, the cash set as the target of the sterilizing processing may be cash temporarily dispensed from the storage unit 14 or may be cash prepared to be supplied to the automatic change machine 1.

With this configuration, since the circulating and conveying unit 123 may not be provided, it is possible to reduce the automatic change machine 1 in size and improve flexibility of disposition of internal components. If the technique according to this embodiment is applied to the automatic change machine 1 according to the second embodiment, the automatic change machine 1 may be configured to enable the user to deposit cash from the dispensing unit 15 as well.

### Fourth Embodiment

In the embodiments explained above, cash is sterilized by irradiating the cash with ultraviolet light. However, not only this, but the sterilization of cash can also be realized by heating. FIG. 10 is a schematic diagram illustrating another example of the configuration of the sterilizing unit 13 illustrated in FIG. 2. The sterilizing unit 13 includes, as illustrated in FIG. 10, an upper-surface heating unit 135 and a lower-surface heating unit 136. The upper-surface heating unit 135 is configured to be capable of heating the upper surface of the cash 50 conveyed in the direction of the arrow 51 on the conveying surface 124. The lower-surface heating unit 136 is configured to be capable of heating the lower surface of the cash 50 conveyed in the direction of the arrow 51 on the conveying surface 124.

Each of the upper-surface heating unit 135 and the lower-surface heating unit 136 is configured to heat the cash 50 on the conveying surface 124 to a predetermined temperature or more. In other words, each of the upper-surface heating unit 135 and the lower-surface heating unit 136 is configured to be capable of generating heat at a predetermined heat flow rate or a predetermined temperature to heat the cash 50 on the conveying surface 124 to the predetermined temperature or more. The temperature of the cash 50 is, for example, surface temperature on a heater side of the cash 50. Naturally, the temperature of the cash 50 may be specified by a center temperature of the cash 50. As each of the upper-surface heating unit 135 and the lower-surface heating unit 136, for example, a heat roller can be used. Generated heat of the heat roller may be Joule's heat or may be heat generated by IH (Induction Heating). Each of the upper-surface heating unit 135 and the lower-surface heating unit 136 only has to be able to generate heat at a predetermined heat flow rate or a predetermined temperature. Heating means other than the heat roller such as a heater using radiant heat may be used.

A degree of the heat generation by the sterilizing unit 13 in the sterilization priority mode only has to be set as appropriate according to, for example, settings of specific heat of each of the upper-surface heating unit 135 and the lower-surface heating unit 136, the materials of bills and coins, and conveying speed such that temperature requested for sterilization corresponding to, for example, types of microorganisms, bacteria, and viruses set as targets of the sterilizing processing is realized on the conveying surface 124.

Generated heat (a predetermined heat flow rate or a predetermined temperature) and conveying speed of the sterilizing unit 13 in the sterilization priority mode are set such that the temperature of the cash 50, which is an object to be heated, is, for example, 70° C. or more. The generated heat and the conveying speed of the sterilizing unit 13 in the sterilization priority mode are preferably set such that the temperature of the cash 50, which is the object to be heated, is, for example, 85° C. or more for one minute or more. The generated heat and the conveying speed of the sterilizing unit 13 in the sterilization priority mode is more preferably set such that the temperature of the cash 50, which is the object to be heated, is, for example, 92° C. or more for fifteen minutes or more. The generated heat of the sterilizing unit 13 in the depositing and dispensing priority mode only has to be set according to conveying speed such that sterilization can be performed in a possible range.

A region on the conveying surface 124 heated by the upper-surface heating unit 135 and a region on the conveying surface 124 heated by the lower-surface heating unit 136 may not coincide.

As explained above, in each of the taking-in and sterilizing unit 131 and the dispensing and sterilizing unit 132, the upper-surface heating unit 135 and the lower-surface heating unit 136 may be integrally configured. The taking-in and sterilizing unit 131 and the dispensing and sterilizing unit 132 may be integrally configured.

Both of the upper-surface heating unit 135 and the lower-surface heating unit 136 do not need to generate heat. A configuration can also be adopted in which at least one of the upper-surface heating unit 135 and the lower-surface heating unit 136 generates heat.

The sterilizing unit 13 can also be a combination of the sterilizing unit 13 according to the first embodiment and the sterilizing unit 13 according to the fourth embodiment. That is, the sterilizing unit 13 can also be configured to irradiate one of the upper surface and the lower surface with ultraviolet light and heat the other.

The sterilizing unit 13 is not limited to the irradiation of ultraviolet light and the heating and only has to be means that can sterilize microorganisms, bacteria, and viruses set as targets of the sterilizing processing. Other means such as being configured to spray a disinfectant can also be used as appropriate.

Such a configuration can also be optionally combined with the embodiments explained above. The same effects as the effects in the embodiments can be obtained by the combination.

### Fifth Embodiment

In the embodiments explained above, all the kinds of cash for each of the denominations discharged from the storage unit 14 are held by the conveying unit 12 in the sterilizing processing in the sterilization priority mode in order to sterilize all the kinds of cash for each of the denominations stored in each of the plurality of storage spaces of the storage unit 14.

Under such circumstances, depending on the capacity of the storage unit 14 and the length of the conveying path of the conveying unit 12, all the kinds of cash for each of the denominations stored in each of the plurality of storage spaces sometimes cannot be held by only the conveying unit 12. Therefore, the automatic change machine 1 according to this embodiment may further include, for example, as illustrated in FIGS. 2 and 4, a temporary storage unit 16 that temporarily holds cash discharged from the storage unit 14. Like the taking-in unit 11 and the storage unit 14, the temporary storage unit 16 includes a separating mechanism 161. The separating mechanism 161 separates, one by one, cash discharged from the temporary storage unit 16. The capacity of the temporary storage unit 16 only has to be set as appropriate according to the capacity of the storage unit 14 and the length of the conveying path of the conveying unit 12.

With such a configuration, in the sterilizing processing in the sterilization priority mode, it is possible to sufficiently sterilize all the kinds of cash stored in the storage unit 14.

In the embodiments explained above, automatic recounting for automatically recounting a balance of cash stored in the storage unit 14 may be executed using, for example, the circulating and conveying unit 123. The automatic recounting can be executed simultaneously with the sterilizing processing in the sterilization priority mode.

In the embodiments explained above, in the depositing and dispensing priority mode, the sterilization time is short because the conveying speed is high. Therefore, the radiation intensity and the generated heat may be set large. Similarly, in the sterilization priority mode, the conveying speed can be reduced, that is, the sterilization time can be increased. Therefore, it is also possible to reduce the radiation intensity and the generated heat and suppress the magnitude of power consumption.

In the embodiments explained above, as an example, the sterilizing processing in the sterilization priority mode is started if the sterilizing instruction from the host machine 9 is received. However, not only this, but, as an example, the sterilization control unit 101 starts the sterilizing processing in the sterilization priority mode according to operation of an operation unit, which is mounted on the automatic change machine 1, by the user. As another example, the sterilization control unit 101 starts the sterilizing processing in the sterilization priority mode at preset time or timing corresponding to a power-off instruction for turning off the power supply of the automatic change machine 1. If the automatic change machine 1 determines whether to start the sterilizing processing in the sterilization priority mode in this way, the storage unit 14 discharges, one by one, the number of pieces of cash of a denomination corresponding to an instruction from the sterilization control unit 101. The conveying unit 12 conveys the cash at speed corresponding to an instruction from the sterilization control unit 101. The sterilizing unit 13 sterilizes the cash at timing and a time width corresponding to an instruction from the sterilization control unit 101.

In the embodiments explained above, as an example, the automatic change machine 1 connected to the host machine such as the POS terminal is illustrated. However, not only this, but the automatic change machine 1 according to this embodiment may be the automatic change machine 1 mounted on a register of a self-service type or a semi-self-service type. In this case, it is possible to eliminate the sanitation concern if not only a store clerk but also a user such as a customer handles cash.

In the embodiments explained above, the automatic change machine 1 connected to or mounted on, for example, the register is illustrated. However, not only this, but the technique according to the embodiment can also be applied to an apparatus that sterilizes a target other than the cash such as game coins.

According to at least one of the embodiments explained above, it is possible to appropriately sterilize cash to be stored.

The control program 1021 executed by the automatic change machine 1 in this embodiment is provided while being incorporated in the ROM or the like in advance.

The control program 1021 executed by the automatic change machine 1 in this embodiment may be provided while being recorded in a computer-readable recording medium such as a CD-ROM, a flexible disk (FD), a CD-R, or a DVD (Digital Versatile Disk) as a file of an installable format or an executable format.

Further, the control program 1021 executed by the automatic change machine 1 in this embodiment may be stored on a computer connected to a network such as the Internet and provided by being downloaded through the network. The control program 1021 executed by the automatic change machine 1 in this embodiment may be provided or distributed through a network such as the Internet.

The control program 1021 executed by the automatic change machine 1 in this embodiment has a module configuration including the units explained above (the sterilization control unit 101 and the storing unit 102). The CPU (the processor) reads out the control program 1021 from the storage medium and loads the units onto a main storage device. Consequently, the sterilization control unit 101 and the storing unit 102 are generated on the main storage device.

The several embodiments are explained above. However, these embodiments are presented as examples and are not intended to limit the scope of the invention. These new embodiments can be implemented in other various forms. Various omissions, substitutions, and changes can be made without departing from the spirit of the invention. These embodiments and modifications of the embodiments are included in the scope and the gist of the invention and included in the inventions described in claims and the scope of equivalents of the inventions.

What is claimed is:

1. An automatic change machine, comprising:
a taking-in component configured to accept cash therein and separate the cash accepted one by one;
a storage component configured to store the cash accepted from the taking-in component, separate a number of pieces of the cash of a denomination corresponding to an instruction one by one, and discharge the cash;
a dispenser configured to dispense the cash discharged from the storage component to an outside;
a conveying component comprising a taking-in and conveying component configured to convey the cash separated one by one by the taking-in component to the storage component and a dispensing and conveying component configured to convey the cash separated one by one by the storage component to the dispenser;
a sterilizing component provided on a conveying path of at least one of the taking-in and conveying component and the dispensing and conveying component and configured to sterilize the cash on the conveying path; and
a controller configured to control a conveying speed of the conveying component to convey the cash at a first conveying speed in a depositing and dispensing priority mode for prioritizing depositing and dispensing and to convey the cash at a second conveying speed slower than the first conveying speed in a sterilization priority mode for prioritizing sterilization of the cash.

2. The automatic change machine according to claim 1, wherein
the conveying component further includes a circulating and conveying component configured to convey the cash on the conveying path of the dispensing and conveying component to the conveying path of the taking-in and conveying component, and
the controller executes, in the sterilization priority mode, sterilizing processing for sterilizing the cash discharged from the storage component and storing the sterilized cash in the storage component.

3. The automatic change machine according to claim 1, wherein the controller executes, in the sterilization priority mode, sterilizing processing for sterilizing the cash discharged from the storage component by reciprocating the cash to pass the sterilizing component on the conveying path on which the sterilizing component is provided of the conveying paths of the taking-in and conveying component and the dispensing and conveying component and storing the sterilized cash in the storage component.

4. The automatic change machine according to claim 1, wherein the sterilizing component comprises an irradiating component configured to irradiate the cash on the conveying path with ultraviolet light having a wavelength in a predetermined ultraviolet region.

5. The automatic change machine according to claim 1, wherein the sterilizing component comprises a heater configured to heat the cash on the conveying path to a predetermined temperature or more.

6. The automatic change machine according to claim 1, wherein the taking-in component comprises at least one of a first intake aperture for coins and a second intake aperture for bills.

7. The automatic change machine according to claim 1, wherein the taking-in component comprises a first intake aperture for coins and a second intake aperture for bills.

8. A method for an automatic change machine comprising:
accepting cash and separating the cash accepted one by one;
storing the cash accepted, separating a number of pieces of the cash of a denomination corresponding to an instruction one by one, and discharging the cash;
dispensing the cash discharged to an outside;
conveying the cash separated one by one to a storage component and conveying the cash separated one by one to a dispenser;
sterilizing the cash on the conveying path; and
controlling a conveying speed to convey the cash at a first conveying speed in a depositing and dispensing priority mode for prioritizing depositing and dispensing and to convey the cash at a second conveying speed slower than the first conveying speed in a sterilization priority mode for prioritizing sterilization of the cash.

9. The method according to claim 8, further comprising:
conveying the cash on the conveying path of a dispensing and conveying component to a conveying path of the taking-in and conveying component, and
using the sterilization priority mode, sterilizing the cash discharged from a storage component and storing the sterilized cash in the storage component.

10. The method according to claim 8, further comprising:
using the sterilization priority mode, sterilizing the cash discharged from the storage component by reciprocating the cash to pass the sterilizing component on the conveying path on which the sterilizing component is provided of the conveying paths of the taking-in and conveying component and the dispensing and conveying component and storing the sterilized cash in the storage component.

11. The method according to claim 8, further comprising:
irradiating the cash on the conveying path with ultraviolet light having a wavelength in a predetermined ultraviolet region.

12. The method according to claim 8, further comprising:
heating the cash on the conveying path to a predetermined temperature or more.

13. The method according to claim 8, further comprising:
accepting cash through at least one of a first intake aperture for coins and a second intake aperture for bills.

14. A POS terminal, comprising:
a registration component; and
an automatic change machine, comprising:
a taking-in component configured to accept cash therein and separate the cash accepted one by one;
a storage component configured to store the cash accepted from the taking-in component, separate a number of pieces of the cash of a denomination corresponding to an instruction one by one, and discharge the cash;
a dispenser configured to dispense the cash discharged from the storage component to an outside;
a conveying component comprising a taking-in and conveying component configured to convey the cash separated one by one by the taking-in component to the storage component and a dispensing and conveying component configured to convey the cash separated one by one by the storage component to the dispenser;
a sterilizing component provided on a conveying path of at least one of the taking-in and conveying component and the dispensing and conveying component and configured to sterilize the cash on the conveying path; and
a controller configured to control a conveying speed of the conveying component to convey the cash at a first conveying speed in a depositing and dispensing priority mode for prioritizing depositing and dispensing and to convey the cash at a second conveying speed slower than the first conveying speed in a sterilization priority mode for prioritizing sterilization of the cash.

15. The POS terminal according to claim 14, wherein
the conveying component further includes a circulating and conveying component configured to convey the cash on the conveying path of the dispensing and conveying component to the conveying path of the taking-in and conveying component, and
the controller executes, in the sterilization priority mode, sterilizing processing for sterilizing the cash discharged from the storage component and storing the sterilized cash in the storage component.

16. The POS terminal according to claim 14, wherein the controller executes, in the sterilization priority mode, sterilizing processing for sterilizing the cash discharged from the storage component by reciprocating the cash to pass the sterilizing component on the conveying path on which the sterilizing component is provided of the conveying paths of the taking-in and conveying component and the dispensing and conveying component and storing the sterilized cash in the storage component.

17. The POS terminal according to claim 14, wherein the sterilizing component comprises an irradiating component configured to irradiate the cash on the conveying path with ultraviolet light having a wavelength in a predetermined ultraviolet region.

18. The POS terminal according to claim 14, wherein the sterilizing component comprises a heater configured to heat the cash on the conveying path to a predetermined temperature or more.

19. The POS terminal according to claim 14, wherein the taking-in component comprises at least one of a first intake aperture for coins and a second intake aperture for bills.

20. The POS terminal according to claim 14, wherein the taking-in component comprises a first intake aperture for coins and a second intake aperture for bills.
